(19) United States
(12) Patent Application Publication
(10) Pub. No.: US 2024/0264145 A1
(43) Pub. Date: Aug. 8, 2024

(54) DEVICE, METHOD, AND SYSTEM FOR THE RAPID DETECTION OF THE TUBERCULOSIS MYCOBACTERIUM DISPERSED IN AIR

(71) Applicants: ALECK ALEXOPOULOS, THESSALONIKI (GR); CHRISTODOULOS SPAGKAKAS, THESSALONIKI (GR)

(72) Inventors: ALECK ALEXOPOULOS, THESSALONIKI (GR); CHRISTODOULOS SPAGKAKAS, THESSALONIKI (GR)

(21) Appl. No.: 18/106,513

(22) Filed: Feb. 7, 2023

(51) Int. Cl. G01N 33/497; G01N 21/64

(52) U.S. Cl. CPC G01N 33/497; G01N 21/6486; G01N 33/4975; G01N 2201/06113

(57) ABSTRACT

A device, method, and system for the rapid detection of the tuberculosis mycobacterium dispersed in air is provided which employ a breath analysis device consisting of a replaceable mouthpiece unit, a laser module, an optical system, and a detection unit capable of detecting autofluorescence generated by the tuberculosis mycobacterium when irradiated by the laser, a software capable of analyzing and characterizing the acquired autofluorescence signal and distinguishing the spectrum due to tuberculosis mycobacteria from spectra generated by other bacteria and providing to the user of the breath analysis device an analysis and report on the results of the breath analysis test.

### FIELD OF THE INVENTION

The present invention relates generally to the field of controlling infectious diseases, detecting bacteria dispersed in air, and detecting infected individuals, and, more particularly to a device, method, and system for the detection of the tuberculosis (TB) mycobacterium.

### BACKGROUND

TB is an infectious respiratory disease with the highest mortality rate worldwide, (e.g., 1.66 m in 2020) affecting all ages with major socio-economic consequences. The total number of people infected with active TB (i.e., which can be transmitted to healthy individuals by inhalation) is about 300m worldwide with 10 m new cases every year. TB can also be present in a non-active latent form (Latent TB Infection or LTBI) which is estimated to infect about one third of the world population. When the immune system is compromised, LTBI can switch to active TB, and the individual can then transmit the disease. According to recent World Health Organization, WHO, reports (2020 and 2021), after a gradual decrease in deaths from 1.8m in 1990 to 1.2 m in 2019, there has been a recent increase in the number of deaths reaching 1.66m in 2020 and even more is estimated in 2021 due to the effects of the covid-19 pandemic. Additionally, there are many TB outbreaks throughout the world and ongoing mutations of TB into new and deadlier strains. Specifically, multiple drug resistant TB (MDR-TB) is usually a result of inadequate care or incomplete therapy. Extensively drug resistant TB (XDR-TB) is an even more drug-resistant strain of TB for which, until very recently, there was no reliable cure. Both these drug resistant forms of TB are increasing as a proportion of total cases.

Treatment of people with TB, and especially those with drug resistant strains of TB, is a lengthy and difficult process with harsh side-effects. Overall, the treatments can last several months and constitute a psychologically and physically traumatic experience for the patients who often cannot endure the full length of the treatment. When patients cease treatment, they are not only promoting the transmission of TB but also increasing the chances of development of even more drug resistant strains.

Due to the co-mingling of populations as a result of increased world-wide travel but also migration and TB co-infection observed in refugees, the risk from active carriers of different strains of TB has increased.

TB is transmitted through the air by droplets, herein called TB-droplets. The TB-droplets may range in size from a few microns to one hundred microns or more and are generated by people with active TB. TB transmission has been found to occur predominantly in crowded closed spaces especially with bad ventilation, e.g., hospitals, prisons, homeless shelters, and other settings, where susceptible persons, i.e., people with an impaired immune system, come in contact with persons with active TB disease. Conceivably, transmission of TB can occur in other crowded conditions, e.g., closed sports stadiums, airports, refugee shelters, military, schools. Transmission occurs by people inhaling dispersed TB-droplets. An untreated person with active TB can infect multiple people, e.g., 10-15, per year. Therefore, in order to decrease the number of new TB cases it is critical that people with active TB be detected and identified rapidly before they can infect others.

The current state-of-art in proposed solutions for TB detection suffer from several problems. Currently the sputum smear microscopy (SSM) method is broadly used in developing nations where TB is prevalent. This method has only a moderate sensitivity of about 50% and even less for people who do not generate sufficient amounts of sputum, e.g., children, those co-infected with HIV. The SSM method requires about 2 hours to provide a diagnosis but may need repeat visits and testing of subjects.

According to WHO, the gold-standard method for TB detection is the culture approach. This approach has high sensitivity, i.e., 95%, and specificity, i.e., 99%, and can also identify drug resistant variants. However, it requires well-trained personnel, special laboratories, and a full diagnosis can take up to 6 weeks. This method is only implemented in developed nations.

Recently, new molecular and immunological detection methods have been developed focusing on the detection of molecules or compounds associated with the TB mycobacterium or the detection of components of the immune system reactive to the mycobacterium (e.g., antibodies). Two such products on the market are the molecular/Polymerase Chain Reaction-based, GeneXpert MTB/RIF® assay, which also tests for resistance to rifampicin, and the immunological-based, QuantiFERON-TB Gold® assay that can identify previous exposure to TB that has produced antibodies thus distinguishing between vaccination, e.g., with the BCG vaccine, or previous exposure to other non-TB mycobacteria. These and other similar methods still present many problems in real world applications and are either difficult to use, unreliable, costly, or are lacking in performance.

Other methods suggested have not been implemented in the field and are also lacking either in ease of use or in performance (i.e., sensitivity, specificity, speed) or cost. These methods include urine testing (e.g., LF-LAM Lipoarabinomannan urine strip test), blood testing and electronic nose (e.g., Aeonose).

Therefore, there is no solution for the point of care diagnosis or detection of TB that can be implemented without previous-training, access to microbiology laboratories, refrigerated storage, or a robust supply of electricity. More specifically, WHO identifies the desired solution as one with high simplicity and performance (i.e., sensitivity, specificity, and speed). Because of the severe limitations on the number of people that can be accurately tested in a short period of time, people infected with TB continue to circulate and transmit the mycobacterium in society.

In many industrial workplaces, e.g., food and cosmetic industries, scanners are used to detect autofluorescence signals from solid surfaces or liquid films. These scanners can rapidly detect bacteria with fluorescent properties but (i) they require large concentrations, (ii) they cannot measure bacteria dispersed in air, and (iii) they do not distinguish the TB bacterium from other types of bacteria.

### SUMMARY OF THE INVENTION

These and other problems of the prior art are solved by the present invention device, method, and system (TBscan) for the detection of the tuberculosis mycobacterium (typically referred to as the TBmb) contained within droplets dispersed in air, i.e., TB-droplets, emitted during exhalation from an infected individual. The device informs the user when the TBmb is detected that the user is infected with active TB and of the risk to transmit the TBmb and infect other individuals.

The present invention provides a breath analysis device for detecting the presence of TB-droplets in exhaled air indicating the presence of active TB in the tested individual. The detection of TB-droplets in exhaled breath is based on the property of autofluoresence and is direct, reliable, instantaneous, and highly sensitive.

The technical difficulty of TB mycobacterium detection from dispersed droplets by laser techniques, e.g., laser induced fluorescence, stems from a number of factors including the typically low concentration of TB-droplets in air, the need for enhanced sensitivity for detecting laser induced fluorescence, the secondary scattering effects at the water-air interface, and the potential effects of the mycobacterium orientation with respect to the direction of laser irradiation. Additionally, the capability to distinguish autofluorescence generated from the TB mycobacterium and other non-TB mycobacteria or other fluorescent entities which influences the specificity of the overall method is a problem which the present invention has overcome.

In an embodiment, a system for detecting TB is comprised of a device for analyzing air from the exhaled breath of an individual and software for analyzing an auto-fluorescent signal obtained from the device. The device employs at least one laser, one detector, and one optic system for detection of laser-stimulated fluorescence from the TBmb. Preferably, the breath analysis device is portable, hand-held, rechargeable, lightweight, and breath-activated to operate at a volumetric flow rate typical of exhalation, e.g., from 30 to 70 liters per minute.

According to an embodiment, the breath analysis device comprises an air inlet filtration unit, a mouthpiece with a separate filter, a flow channel, a detection section, a laser source, an optical system, a detector unit, and a power source. The software detects the presence of an autofluoresence signal and then analyzes the obtained autofluoresence spectrum to certify that it is generated by the TBmb. In an embodiment, the software is implemented as a mobile application for a smart phone, lap top, and the like. In another embodiment, the software is integrated with the breath analysis device.

In operation, exhaled air from a user who is being tested enters into the device from the mouthpiece unit through a coarse filter permitting only small droplets (e.g., <50 μm) to enter the flow channel. Ambient air also enters the device after passing through a fine filter eliminating all dispersed droplets and particles greater than 1 μm. The inflowing ambient air surrounds the inflowing exhaled air preventing exhaled droplets from depositing onto the interior surfaces of the device.

The air in the flow channel passes by a laser activation sensor which activates the downstream laser module. The activated module emits a monochromatic laser of a wavelength, e.g., in the 320-400 nm range, capable of eliciting autofluorescence from the TBmb. The laser module is activated for a period of time equal to the duration of a single exhalation, for example, 4-6 seconds. The average laser power, which should be sufficient to stimulate the mycobacteria so that they emit autofluorescence light at a larger wavelength, is for example between 0.5-2 mW. These conditions are favorable for creating autofluoresence light from TB-droplets which is emitted between the wavelengths of 390 and 460 nm. The autofluorescence light reaches and activates a detector (with sufficient sensitivity, e.g., minimum light current, to generate a signal from incident light of different wavelengths) generating a detectable autofluorescence signal. When the autofluorescence signal is above a specific threshold, for example, but not limited to signal peak above 5% of the baseline, an LED light on the device is activated and a signal reporting this event is sent to a receiver, e.g., an application in a smart phone, and the like.

More specifically, according to an embodiment of the present invention, the breath analysis device collects autofluorescent spectra, processes, and then transmits these by WiFi as digitized signals to a mobile phone or other personal device (e.g., tablet, laptop). The transmitted signals are processed by software and the results are displayed locally on the mobile device and may also be communicated back to the breath analysis device and indicated by the LED light as a confirmed detection. The system also includes software on a mobile phone or tablet application which provides outputs of the breath analysis device such as status, number of droplets analyzed, number of potential positives, number of definite positives (as validated later with other diagnostics), autofluorescence spectra averaged over increments of time, for example but not limited to 60 seconds, as well as spectrum analysis for validation of the emitter identity.

The present invention system and device are advantageous over the existing solutions because they provide instant detection of the TB mycobacterium, thus identifying active TB carriers, and it minimizes the risk of TB transmission and infections. The present invention is very easy to use requiring only directions for changing the mouthpiece after each use. The sensitivity of this solution is better than the SSM method and similar to the molecular approaches. Therefore, this invention provides an easy to use, instant, cheap, and sensitive solution for detecting people infected with TB in all locations including emerging economy nations with minimal resources and trained personnel.

The various embodiments of the present invention providing detection, warning, and identification system and device offer elegant and simple solutions for detecting TB and deliver a number of advantages and benefits including but not limited to the following:
(i) The main embodiment of the invention is effectively a point of care solution for the diagnosis of TB. This enables the broad testing of large groups of people to minimize the spread of TB.
(ii) Infected people are protected by rapid testing for TB that leads to early isolation and treatment. Early isolation reduces the chances of transmission and early treatment increases the outcomes of medication.
(iii) The general public benefits by providing protection to people against infection both by identification and isolation of infected people but also by providing early warnings of closed spaces which has become contaminated with TB-droplets.

The present invention is positioned well beyond the state of the art of methods and devices for detecting people with active TB. The present invention is comprised of a new device which can detect the presence of the TB mycobacterium in dispersed droplets through sensitive autofluorescence detection.

The present invention allows for the point-of-care detection of the TBmb in air exhaled from an individual which is characterized by its sensitivity, specificity, and rapidity of detection. Heretofore, it is believed to be the only method able to directly detect TB mycobacteria dispersed in air droplets exhaled from an infected individual. The direct detection of the TBmb by this method is advantageous over other indirect methods that attempt to achieve detection by examination of the physiological effects of TB infection, e.g., analysis of volatile organic compounds in exhaled breath.

The low-cost and ease-of-use of this invention make it attractive for detecting TB in emerging economy nations and superior to existing methods that require trained personnel, well-equipped microbiology laboratories, stable electricity, and product support.

The present disclosure describes a system and method that solve the aforementioned drawbacks in prior art solutions in terms of monitoring and detecting people with active TB.

These and other advantages of the present invention system and method will become better understood by those skilled in the art of the present invention from the following detailed description in conjunction with the accompanying drawings.

### BRIEF DESCRIPTION OF THE DRAWINGS

By reference to the appended drawings, which illustrate exemplary embodiments of this invention, the detailed description provided below explains in detail various features, advantages and aspects of this invention. As such, features of this invention may be more clearly understood from the following detailed description considered in conjunction with the following drawings. The exemplary embodiments illustrated in the drawings are not intended to be considered limiting of the scope of the invention for the invention may be implemented with other equally effective embodiments.

FIG. 1 is a schematic of the TBscan system. The TBscan breath analysis device 11 consists of the removable mouthpiece 12, the on-off switch 13, and the LED indicator 14. The smartphone 15 is shown running the TBscan software 16, as well as the server 17 where all the testing data is stored.

FIG. 2 is a schematic of a breath analysis device generally designated with numeral 21. The device 21 measures the presence of autofluorescent TB or non-TB mycobacteria in exhaled air. The exhaled air may be exhaled from an individual who is being tested.

The breath analysis device 21 comprises an activation sensor (AS), 22, a laser module (LM), 23, an electrical connection between the LM and the AS, 214, an autofluoresence detector, (AFD) generally designated with numeral 24 and an optical system consisting of a reflector mirror (RM), 25, a collector lens (CL), 26 and a scattered light filter (SLF), 27. The device also consists of a removable mouthpiece unit (MU), 28, situated at the air inlet of the device 21. The MU includes a coarse inlet filter (F1), 29, at an inlet thereof. An annular inflow opening, 210, is formed between the MU, 28, and the inlet of the device 21 with a ring-shaped fine filter 211 positioned therein. The air inlet leads to the flow channel (FC), 212, which then leads to the detection section (DS), 213.

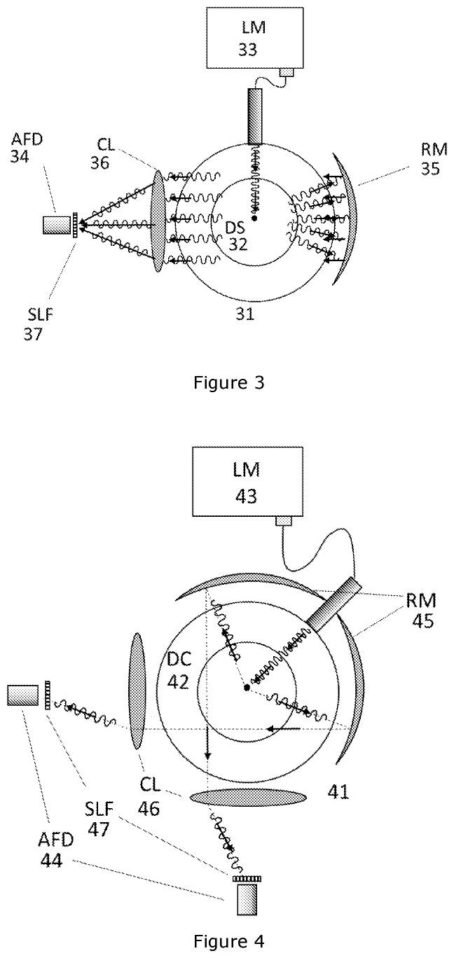

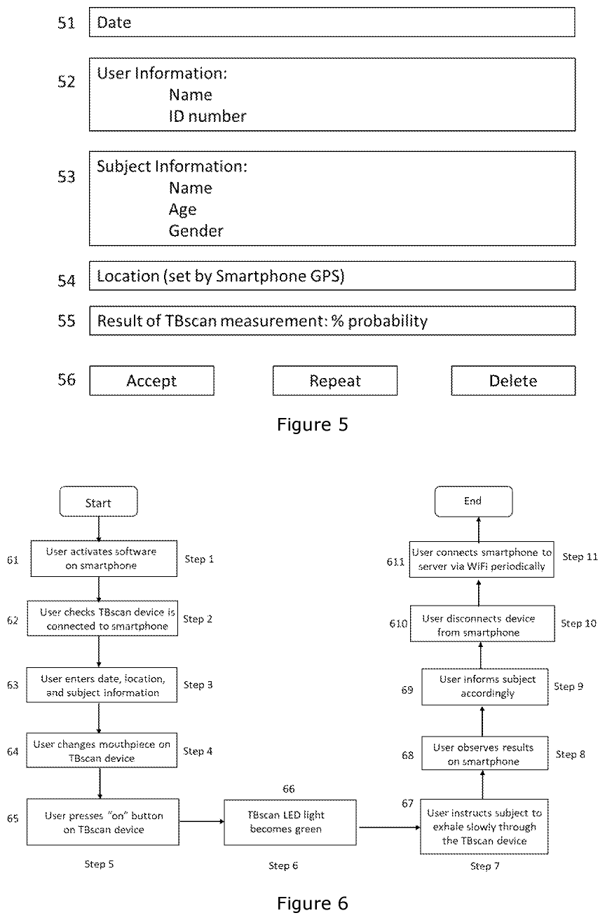

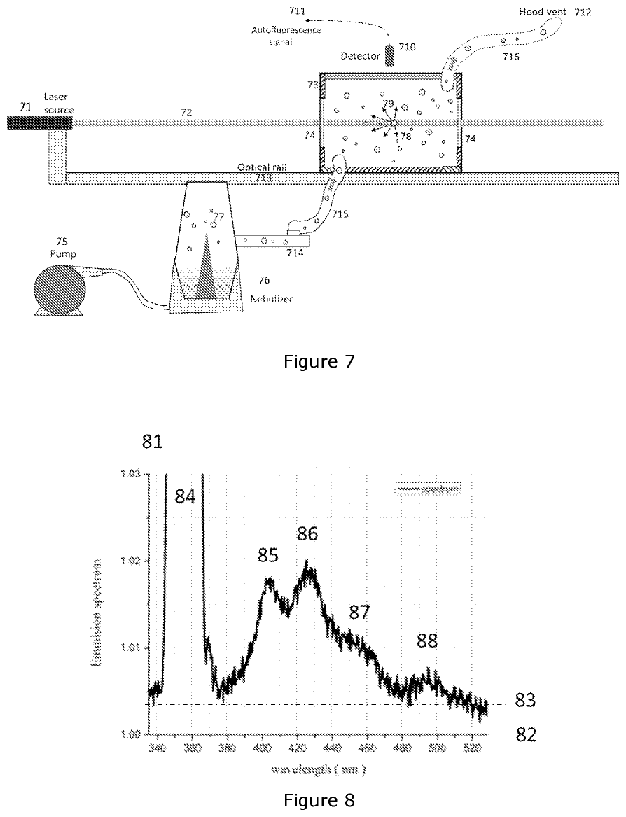

FIG. 3 is a schematic of the device, 31, at a cross-section situated at the detection section (DS), 32, of the device of FIG. 2 and depicts the laser source module (LM), 33, and the position and orientation of the AFD, 34, the RM, 35, the CL, 36, and the SLF, 37.

FIG. 4 is a schematic of another embodiment of the breath analysis device, 41, at a cross-section situated at the detection section DS,42, of the device of FIG. 2 and depicts the laser source module LM, 43. This embodiment consists of a dual detector and optical system whose position and orientation are indicated in the Figure. It consists of two autofluorescence detectors (AFD), 44, two reflecting mirrors (RM), 45, two collector lens (CL), 46, and two scattered light filters (SLF), 47.

FIG. 5 is a schematic of the TBscan software interface. The main information provided therein includes the date of testing, 51, the user information (name and ID number), 52, the name, gender, and age of the subject being tested, 53, the location, 54, the results of the TBscan test, 55, and an acceptance or rejection option panel, 56.

FIG. 6 is a flowchart provided the step-by-step actions of the user when testing a subject who might be infected with TB over a sequence of 11 steps.

FIG. 7 is a schematic of a proof-of-concept experiment for the detection of autofluorescence light emitted by a TB-analogue. A laser source 71 situated on an optical rail 713 generates a laser 72 which enters an environmental chamber 73 through quartz windows 74. A pump 75 feeds a nebulizer 76 generating aerosol droplets of water 77 containing the TB-analogue. The contents of the nebulizer exit through a modified outlet 714 and are directed to the chamber by a connecting tube 715. The laser stimulates the TB-analogues 78 to emit autofluorescent light 79 which reach the detector 710 generating an autofluorescent signal. The contents of the environmental chamber are released to a hood vent 712 through a connecting tube 715.

FIG. 8 is a graph of the autofluorescent signal obtained in the proof-of-concept experiment depicted in FIG. 7. The autofluorescent signal is presented in the graph as signal intensity 81 with respect to wavelength of light 82. Compared to the baseline 83 one can identify the peak corresponding to scattered light from the stimulating laser 84 and several peaks 85, 86, 87, 88 which are due to the autofluorescent light.

### DETAILED DESCRIPTION

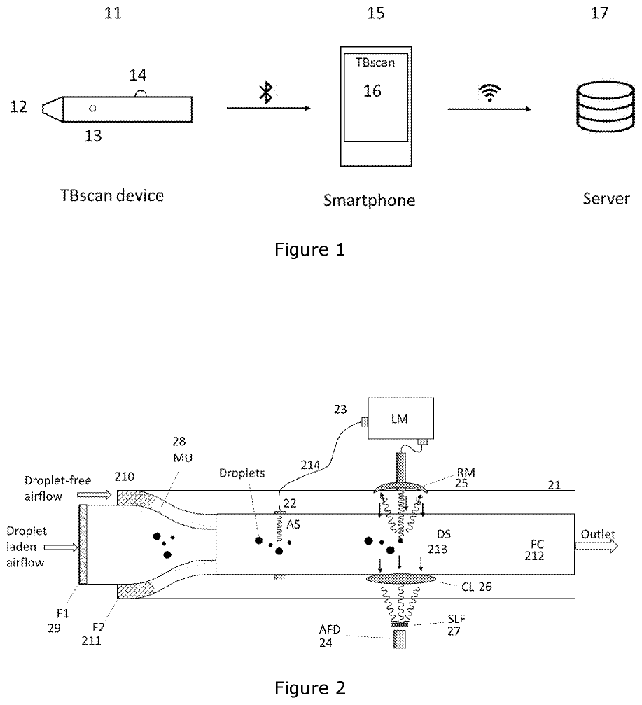

Referring to FIG. 1, there is provided a TBscan system for measuring the presence of autofluorescent entities of the tuberculosis mycobacterium in the exhaled air of a subject. The TBscan system consists of a breath analysis device, 11, which communicates with a smartphone, 15, and data is stored on a secure server, 17. The user initiates a TB diagnostic test using the TBscan system by first changing the removable mouthpiece, 12, of the device before each test. The device is activated by the user by an on-off switch, 13, and the subject exhales into the device when the LED indicator, 14, turns green. The user utilizes a smartphone, 15, running the TBscan software, 16, which is part of the TBscan system. The user enters data for each subject being tested into the software interface. After the exhalation test of each subject, the user reads the results of the test on the TBscan software, and proceeds accordingly. The test specific data including the results are sent to a server, 17, where a database of all the testing data is stored.

Referring to FIG. 2, there is provided a breath analysis device, 21, for measuring the presence of autofluorescent entities of the tuberculosis mycobacterium in the exhaled air of a user. The device comprises of at least one activation sensor AS, 22, for the detection of the initiation of exhalation from the user. The activation sensor sends a signal to activate the laser module LM, 23, and autofluoresence detector AFD, 24, systems downstream through an electrical connection, 214. In an embodiment, the LM consists of a continuously operating monochromatic laser at a wave length of, for example, 360 nm with a cross-section of 2 mm and an average power of 1 mW. In this embodiment, the continuously operating laser can be used intermittently when an entity dispersed in air is detected upstream by the activation sensor. In an embodiment, the AFD consists of a static diffraction grating element and a charged-coupled device, CCD, camera as an array detector of photons.

The AFD detects autofluorescence light emitted by the dispersed entities after laser-stimulation and direction of the autofluorescence light to the detector and concentration onto the aperture of the detector by an optical system. The optical system consists of a curved reflector mirror RM, 25, which collects emitted autofluorescent light-waves and delivers them to a concave or semi-concave collector lens CL, 26, which then focuses the incoming light onto the aperture of the AFD, 24, after passing through a scattered light filter SLF, 27. The SLF is set at wave-length cut-off just above the wavelength of the laser, for example, 370 nm. The purpose of the filter is to improve the sensitivity of the AFD by eliminating the stronger signal of the scattered laser light from reaching the detector and washing-out the much less intense autofluorescence signal.

The device also consists of a removable and disposable mouthpiece unit MU, 28, situated at the air inlet of the device that is replaced after every use. The mouthpiece unit includes a coarse inlet filter F1, 29, and an annular inflow opening, 210, through which ambient air enters the device enveloping the airflow generated by the exhaled breath of the user. The F1 filter removes large droplets (e.g., greater than 50 μm in size) in exhaled breath which could otherwise settle due to gravity and deposit in the device. The inflow opening passes through a fine filter F2, 211, that removes both fine and coarse particles and droplets (e.g., greater than 5 μm in size) generating a filtered stream of ambient air that envelops the exhaled airstream from the MU entering the flow channel, 212, of the device. This prevents direct contact of droplets in the exhaled breath with the interior surfaces of the device. In this way cross-contamination between users is avoided. To ensure minimal radial mixing, the flow is kept within the laminar flow regime either by an increased cross-sectional area or an increased filter resistance. Both these interventions reduce the velocity of airflow and maintain a laminar flow pattern.

The airflow from the MU enters the main flow channel FC, 212, and passes by the AS, 22, before entering the detection section DS, 213, where the main laser illuminates the dispersed droplets and elicits autofluorescence from entities within the droplets. The autofluorescence detected by the AFD is sent in digital form via bluetooth to the TBscan software where it is further processed.

Referring to FIG. 3, there is provided a 90-degree rotated view of the device, 21, shown in FIG. 2. The device is shown at a cross-section of the axis of the air flow path at a plane intersecting the detection section DS, 32, the laser source module LM, 33, and the AFD, 34, of the device. This orientation of the device is presented to display the path of the autofluorescence signal in the DS from an autofluorescent entity to the reflecting mirror RM, 35, to the collector lens CL, 36, through the scattering light filter SLF, 37, before reaching the AFD. When an autofluorescent entity, e.g., a TB-droplet, located on the axis of symmetry of the flow chamber, is stimulated by the laser, autofluorescent light is emitted at different angles. The light reaching the RM is reflected in parallel toward the CL which directs the light towards the AFD whose aperture is located at the focal length of the CL. The curvature, radius, and position of the RM is designed to obtain and reflect a maximum signal to the CL from autofluorescence generated from a volume of air stimulated by a laser of a specific diameter, for example 1.5 mm.

Referring to FIG. 4, there is provided a 90-degree rotated view of the device 21 shown in FIG. 2. This device is another embodiment of the invention with a dual optical detection system. The device is shown at a cross-section of the axis of the air flow path at a plane intersecting the detection section DS, 42, the laser source module LM, 43, and two autofluorescence detectors AFD, 44, of the device. In this embodiment of the device there are two optical systems consisting of two reflecting mirrors RM, 45, two collector lens CL, 46, two scattered light filters SLF, 47, and two fluorescence detectors. The laser source is situated between the two RM. This embodiment provides double the autofluorescence signal and sensitivity compared to the embodiment with a single AFD, RM, CL, and SLF system.

Referring to FIG. 5, the TBscan software interface is shown. The interface is designed to be very easy to use with only essential information. The user (e.g., health care worker, volunteer) enter their name and ID number (if relevant), 52, the name, gender, and age of the subject being tested, 53. The smartphone automatically completes the date and time, 51, and enters a location, 54, which the user can edit. After the test, the software provides a probability estimate, 55, that the subject is infected with active TB. The user decides if the test is acceptable or not or if it should be repeated and enters their decision into the software, 56.

In another embodiment of the software, e.g., for clinical trials and research applications, additional information will be provided to the user regarding the autofluorescence spectra, spectra characteristics (e.g., number and wavelength of spectra peaks) signal processing analytics (e.g., area under curve, signal to noise ratio), and other information of relevance (e.g., environmental conditions such as temperature and humidity).

Referring to FIG. 6, a user flowchart is provided indicating the step-by-step actions of the user when testing a subject who might be infected with TB. The user first activates the TBscan software on their smartphone 61 and then turn on bluetooth and checks that the TBscan device is connected to their smartphone 62. The user then enters the location (if not automatically set), their name and ID number, and the information of the subject about to be tested, i.e., their name, age, and gender 63. The user then replaces the mouthpiece of the TBscan device preparing for the subject test 64. The user then presses the “on” button 65, waits until the LED indicator becomes green 66, and then instructs the subject to exhale slowly through the mouthpiece into the TBscan device 67. The user then observes the results of the test on their smartphone 68, decides if the test should be accepted or repeated, and informs the subject accordingly 610. At the end of the testing session the user disconnects the TBscan device from the smartphone and removes the mouthpiece 611. When internet access is possible the user can turn on the device which will automatically update a database on a secure server 612.

Referring now to FIG. 7, a schematic of a proof-of-concept experiment for the detection of autofluorescence light emitted by a TB-analogue is provided. The TB-analogue in this experiment were spherical polystyrene particles, 2 μm in size, with a coating with similar autofluorescent properties as the actual TBmb (Sigma L0280-1 ml, amine-modified PS fluorescent blue). A laser source 71, situated on an optical rail 713, generates a laser 72 of wavelength of 350 nm which enters an environmental chamber 73 of size 15×15×10 cm, through quartz windows 74 transparent to the laser wavelength. A pump 75 feeds a modified commercial nebulizer 76 generating aerosol droplets of water 77, up to 5 μm in size, containing the TB-analogue. The contents of the nebulizer, i.e., a fine mist of droplets containing the TB-analogues, exit the nebulizer through a modified outlet 714 and are directed to the environmental chamber through a connecting tube 715. The laser stimulates the TB-analogues 78 crossing the laser path-line in the environmental chamber which emit autofluorescent light 79 which reaches the detector 710 generating an autofluorescent signal. The contents of the environmental chamber are sent to a hood vent 712 through a connecting tube 716.

Referring now to FIG. 8, a graph of the autofluorescent signal obtained in the proof-of-concept experiment depicted in FIG. 7 is provided. The autofluorescent signal is presented in the graph as signal intensity 81 (y-axis) at different wavelengths of light 82 (x-axis). From the full range of wavelengths, baseline can be identified 83. Compared to this baseline one can identify the peak corresponding to scattered light 84 from the stimulating laser and several peaks 85, 86, 87, 88 which are due to the autofluorescent light generated by the TB-analogue.

Several proof-of-concept experiments were performed to validate reproducibility of the obtained autofluorescent signal and to determine the sensitivity limits for the specific experiment shown in FIG. 7. From these experiments one concludes that
The autofluorescent signal generated by the TB-analogue dispersed in water droplets which are dispersed in air can be detected.
The autofluorescent signal can be analyzed as a spectrum of different intensity peaks situated at different wavelengths characteristic of the entity emitting autofluorescent light
The sensitivity limit of this experiment, considering also improvement of the optical system and inclusion of filters to eliminate the dominating scattered light signal, can be determined to be sufficient to detect an autofluorescnce signal from the TBmb in the exhaled breath of infected individuals.

This embodiment can be used in hospitals by medical professionals or even as a point of care solution by healthcare workers with minimal training or guidance. The operation of the device is simple and intuitively obvious and the mouthpiece can be readily changed between uses. Compared to existing solutions the TBscan system is vastly superior in broad scale scanning of populations of emerging economy nations as it is a fast, easy, and cheap point of care solution that does not require specialized laboratories or trained technicians. In established economy nations it can become part of a general patient screening, evaluation and check-up.

In another embodiment of the invention, a pulsed laser, for example but not limited to, a neodymium-doped yttrium aluminum garnet laser operating at 1 mW with 50 fs pulses at 1 MHz, can be used instead of a continuous laser. The pulsed laser can be selected to provide optimal detection characteristics and power consumption.

In another embodiment of the invention, multiple identical laser sources are employed to illuminate a larger volume in the detection section in order to stimulate autofluorescence from a larger number of droplets and thus increase signal intensity and sensitivity.

Also, for example, the system of claim may employ two lasers of different wavelengths either at the same position or at different locations. This dual stimulation system will generate different autofluorescence spectra and provide extra information which will improve sensitivity and selectivity.

In another embodiment of the device two activation sensors in series, oriented at a 90-degree angle with respect to each other, are employed to detect a dispersed droplet moving along the centerline.

In another embodiment the mouthpiece device can have different geometry than what is indicated in the main embodiment, e.g., conical, or filter position, e.g., at the mouthpiece outlet, compared to that indicated in FIG. 2. Mouthpieces can be designed to minimize air flow resistance and employed for subjects unable to generate the necessary air flow through the device, e.g., for individuals with respiratory impairments such as children, and elderly subjects.

In another embodiment of the invention the breath analysis device employs a fan situated at the outlet of the device to provide an increased air flow rate (e.g., from 5-50 times more) and a different mouthpiece with a more restrictive filter (e.g., less than 20 μm). This embodiment of the device enables applications where the source of TB-droplets is removed from the device, for example by 1-2 m. Examples of such applications include the monitoring of individuals seated across a desk, e.g., in a doctor's office setting as well as the screening of travelers in a full body scanner unit in airports.

Although the invention is described in terms of specific embodiments, it should be understood that other embodiments and variations thereof may be envisioned by the skilled person in the art without departing from the scope of the invention as defined in the following claims.

What is claimed is:

1. A system for detecting TB mycobacteria in droplets dispersed in an air sample exhaled by a user, through a disposable mouthpiece, into a breath analysis device, connected to an App, processing autofluorescence signal data and communicating the results to the user, and a server, storing each subject's test results (FIG. 1).

2. The system of claim 1 comprising a breath analysis device with
a disposable mouthpiece
a flow channel with optically transparent walls.
The flow channel is comprised of
an activation section,
a detection section.
The activation section consists of an activation sensor which is a low power laser, continuously operating while the device is on, which detects the presence of droplets that are approaching the detection section and activates the laser module.
The detection section of the flow channel consists of
a laser module configured to generate an autofluoresence signal from droplets containing the TBmb passing through a laser path-line in the detection section. The laser module includes a continuously operating laser at a range between 300 and 420 nm, preferably between 320 and 400 nm, and more preferably from 340 to 380 nm. The laser dimensions may range, for example, from 1 to 2 mm. The continuously operating laser has a power of at least 0.5 mW and operates over a time period of 1 to 10 s.
a reflecting mirror situated outside the detection section for collecting the autofluorescence light and for concentrating this light to the collector lens,
the collector lens for focusing the collected light onto a detector aperture after passing through a scattered light filter
the scattered light filter which blocks the high intensity scattered laser light from entering and saturating the detector with a wavelength cut-off typically 10 nm more than the monochromatic laser wavelength,
the autofluorescence detector unit which generally consists of a photon dispersive unit, e.g., a static diffraction grating element, and a photon detection unit, e.g., a silicon photodiode, PIN, or an array detection unit or spectroscopy camera such as CCD, CMOS, with the necessary sensitivity (e.g., minimum light current of 10 mA at 0.5 mW/cm²) configured in a way to minimize the effect of scattered photons and to achieve the desired resolution of the fluorescent spectrum.
as indicated in FIG. 2.
In the main embodiment the optical detection system (consisting of at least one reflecting mirror, at least one collection lens, at least one scattered light filter, and at least one detector) is capable of detecting at least 20% of the autofluorescence generated by laser stimulation (FIG. 3).

3. The system of claim 1, wherein exhaled air from the subject being tested enters the breath analysis device through a disposable mouthpiece, which is replaced after each use. The mouthpiece contains a filter for coarse droplets (e.g., at least 50 μm) which can foul the interior of the device. Smaller droplets (e.g., less than 50 μm) pass through the mouthpiece filter and enter the detection section. The mouthpiece shape is designed in a way to keep the inhaled air away from the interior surfaces of the detection chamber and avoid deposition and fouling. The air leaving the mouthpiece is directed parallel to the detector chamber surfaces at a low enough velocity that turbulence is avoided (e.g., Reynolds number less than 1500). Surrounding air also enters the breath analysis device and envelops the inflowing stream of exhaled breath. The inflowing surrounding air passes through a fine filter element eliminating particles and droplets (e.g., greater than 5 μm), which is replaced after a certain number of uses, e.g., 100, which depends on the quality of surrounding air.

4. The system of claim 1, wherein the device further comprises an LED light to provide the status of the device and the result of the test to the user (e.g., green signifying the device is ready for use, blue signifying a negative detection result, and red signifying a positive detection result) and means of signal transmission and reception by WiFi to a smartphone.

5. The system of claim 1, wherein software collecting and processing data being transmitted by WiFi from the breath analysis device is installed on a smartphone (or tablet or device thereof). The software employs a user-friendly interface where the user (e.g., health care worker, volunteer) enters relevant information for each test. After executing a test, the user receives through the interface the results of the test. The software also transmits to the device the results of the test which the device displays through the LED light (i.e., blue and red indicating negative and positive detection results, respectively). The software also transmits the results to a safe and secure server through the internet.

6. The system of claim 1, wherein a safe and secure server collects and organizes in a database data from each test including data regarding the subject being tested (pseudo-anonymized), the user executing the test (pseudo-anonymized), the location, date, and environmental conditions at the test site, as well as the test results. This data is typically collected for statistical purposes and to evaluate the performance of the TBscan solution.

7. The system of claim 2, further comprising multiple optical elements and detectors to increase the total collected autofluorescence signal. For example, a dual system with two reflecting mirrors, two collecting lens, two scattering light filters, and two detectors will double the signal intensity and sensitivity (FIG. 4). By employing a dual optical detection system, the detection efficiency is at least 40%.

8. The system of claim 2, further using multiple identical laser sources illuminating a larger volume in the detection section in order to stimulate autofluorescence from a larger number of droplets and thus increase signal intensity and sensitivity.

9. The system of claim 2, wherein two lasers of different wavelengths are employed either at the same position or at different locations, for generating different autofluorescence spectra to provide extra information for improved sensitivity and selectivity.

10. The system of claim 2, further using an axial orientation of the laser with radial placement of optical elements and detectors for increasing the volume illuminated by the laser and increase the autofluorescence signal and the sensitivity of the system.

11. The system of claim 2, wherein a pulsed laser is employed instead of a continuous laser. The pulsed laser, for example, a neodymium-doped yttrium aluminum garnet laser operating at 1 mW with 50 fs pulses at 1 MHz, can be typically selected to provide optimal detection characteristics and power consumption.

12. The system of claim 2 wherein the breath analysis device is turned on manually but the laser module is in stand-by mode. The laser module is only activated when the activation sensor detects an entity in air after exhalation is initiated by the subject being tested.

13. The system of claim 2, wherein multiple activation sensors, e.g., oriented at different angles with respect to each other, are employed to detect a dispersed droplet moving along the centerline of the flow channel, and whereby the droplets on the centerline are best positioned to provide the maximum autofluorescence signal.

14. The system of claim 2, wherein the mouthpiece employs a different geometry, e.g., conical, or filter position, e.g., at the mouthpiece outlet, compared to that indicated in FIG. 2.

15. The system of claim 2, wherein mouthpieces designed to minimize air flow resistance are employed for subjects unable to generate the necessary air flow through the device, e.g., for individuals with respiratory impairments such as children, and elderly subjects.

16. The system of claim 5, wherein the software presents details of the signal analysis of collected autofluoresence spectra and calculations to the user. This software version could be for example intended for research purposes and could enable changes in some parameters related to signal analysis and the criteria for TBmb detection. For example, differentiating criteria in terms of the number of peaks, peak position, sensitivity criteria in terms of area under the curve and peak height, spectra categorization based on spectra shape broadness, skewness, and modality.

17. The system of claim 5, wherein the TBmb identification software is modified to be further capable of detecting certain other mycobacteria with autofluorescence properties (for example mycobacteria of the avium complex, e.g., Mycobacterium abscessus) by advanced signal analysis and differentiation of the autofluorescence spectra.

18. The system of claim 2, wherein the breath analysis device employs a fan situated at the outlet of the device and a different mouthpiece with a more restrictive filter (e.g., less than 20 μm). This version of the device can be implemented for longer periods of time than a single exhalation or even continuously. This version could also be used with individuals with impaired respiratory capacity (e.g., children, elderly) to assist in airflow through the device.

19. The system of claim 18, wherein a device is used as a desktop monitor of exhaled breath from an individual seated in close proximity, e.g., 1-2 m from the emitting infected individual. In this embodiment, the device would be in continuous operation collecting data over a longer period of time than an exhalation, for example, 1-10 minutes, but still providing the same sensitivity as in the test of a single exhalation of the main embodiment in claim 1. This system could be used, for example, in doctor office settings to obtain a warning signal.

20. The system of claim 18, wherein the device is incorporated into an airport security screening procedure of travelers, for example, full body scanner units. In this embodiment, the device would be in continuous operation during the scanning test of a subject collecting data over a longer period of time than an exhalation, for example, up to 2 minutes, but still providing the same sensitivity as in the test of a single exhalation of the main embodiment in claim 1.
